(19) United States
(12) Patent Application Publication
(10) Pub. No.: US 2026/0346213 A1
(43) Pub. Date: Nov. 5, 2026

(54) COUPLING ELEMENT FOR COUPLING A DRIVE ELEMENT TO A STIRRING UNIT FOR A FLUID CONTAINER, SYSTEM, AND USE

(71) Applicant: RHEINISCH-WESTFÄLISCHE TECHNISCHE HOCHSCHULE AACHEN ABGEKÜRZT RWTH AACHEN KÖRPERSCHAFT DES ÖFFENTLIC, Aachen (DE)

(72) Inventors: Patrick Bongartz, Neuss (DE); Moritz Meyer, Pulheim-Dansweiler (DE); Kai Thesing, Aachen (DE); Christian Kneppeck, Aachen (DE)

(21) Appl. No.: 19/486,022

(22) PCT Filed: May 16, 2024
(86) PCT No.: PCT/EP2024/063526

§ 371 Date: Nov. 19, 2025

(30) Foreign Application Priority Data
May 24, 2023 (DE) 10 2023 113 536.8

(51) Int. Cl. B01F 23/233; B01F 35/32; B01F 101/00

(52) U.S. Cl. CPC B01F 23/23311; B01F 23/23314; B01F 35/3214; B01F 2101/2204

(57) ABSTRACT

In an embodiment a system includes a coupling element configured for coupling a drive element to a stirring unit, wherein the coupling element includes a base body, wherein the base body has a first fluid path configured for conveying a first fluid and at least one second fluid path configured for conveying a second fluid, wherein the first fluid path and the at least one second fluid path are separated from each other in terms of fluid flow, wherein the base body comprises an outer part with an interior space and a middle part with a first cavity and with at least one second cavity, wherein the middle part has a first connecting element configured for transmitting a driving force from the drive element and a second connecting element configured for transmitting the driving force to the stirring unit, and wherein the middle part is rotatably mounted to the outer part.

### CROSS-REFERENCE TO RELATED APPLICATIONS

This patent application is a national phase filing under section 371 of PCT/EP2024/063526, filed May 16, 2024, which claims the priority of German patent application no. 10 2023 113 536.8, filed May 24, 2023, each of which is incorporated herein by reference in its entirety.

### TECHNICAL FIELD

The present invention relates to a coupling element for coupling a drive element to a stirring unit. The present invention further relates to a system with a coupling element and a stirring unit. The present invention further relates to a method for operating a coupling element.

### BACKGROUND

In chemistry or biology, for example for microbiological investigations or cell cultures, substance transfers between fluids are frequently required. Examples of such substance transfers are a transfer of O₂ from air into water or a transfer of a dissolved, non-polar product of an upstream chemical reaction into an extraction liquid. A transfer of substances is usually caused or at least facilitated by bringing the substances involved in the transfer into contact with each other, for example by stirring, blending, dispersing, or similar. For certain processes, it is intended that a gas be introduced into a liquid in the form of bubbles, for example to enable biosynthesis.

The liquid can be given into a so-called bioreactor and the gas can be introduced using a gasification and stirring unit immersed in the liquid. The gasification and stirring unit can be configured as a movable unit with membrane elements, whereby the membrane element is intended for the diffusive or convective transfer of a first fluid, for example a gas or a liquid, into another fluid. A gasification and stirring unit can be used to optimize the distribution of the first fluid in the other fluid, whereby the occurrence and size of bubbles can also be controlled. Such a gasification and stirring unit is known, for example, from publication WO 2021/152128 A1.

However, when using the known gasification unit, the drive and fluid delivery to or from the gasification and stirring unit remain a challenge, in particular because these usually have to take place simultaneously. If known laboratory equipment for gas supply and other means of driving the gasification and stirring unit are assembled and combined, the result is a connection technology that requires a relatively large amount of space. It is also difficult to ensure a secure connection between the various components.

Another challenge is retrofitting existing reactors or fluid containers with the known gasification and stirring unit. As a rule, fluid containers or reactors are already equipped with a drive element for a movable part, for example a simple stirring unit, whereby software is provided for controlling the drive. For retrofitting or replacing the simple stirring unit with the known gasification and stirring unit, a complex and cost-intensive replacement of the existing drive including software has been carried out up to now.

### SUMMARY

Embodiments of the invention provide improved connection means for the simultaneous transmission of a drive force and for the transmission of at least two fluids for a stirring unit.

Embodiments of the invention provide a coupling element for coupling a drive element to a stirring unit, wherein the coupling element comprises a base body, wherein the base body has a first fluid path for conveying a first fluid and at least one further fluid path for conveying a further fluid, wherein the first fluid path and the at least one further fluid path are separated from each other in terms of fluid flow, wherein the base body has an outer part with an interior space and a middle part with a first cavity and at least one further cavity, wherein the middle part has a first connecting element for transmitting a drive force and a second connecting element for transmitting a drive force, wherein the middle part is rotatably mounted to the outer part, wherein the middle part is at least partially arranged in the interior of the outer part and the interior is configured such that a free area of the interior surrounds the middle part, wherein the free area of the interior of the outer part and the first cavity of the middle part together at least partially form the first fluid path, and wherein the at least one further cavity of the middle part at least partially forms the at least one further fluid path.

Such a coupling element enables both the transmission of force from a drive element to a stirring unit and the transmission of at least two fluids simultaneously.

The rotatable mounting of the inner part with the outer part on the one hand, and the respective shaping of the outer part and the inner part and their arrangement relative to each other, which form the first fluid paths, on the other hand, ensure a rotation-independent connection of a fluid source or disposal unit to a stirring unit. This eliminates the need for so-called rotary locks for connecting, for example, fluid sources to the coupling element. In addition, the coupling element can be configured to be compact, which is particularly advantageous for retrofitting.

According to embodiments of the invention, the aforementioned task is further solved by a system comprising a coupling element according to the present disclosure and a stirring unit, wherein the coupling element has a connecting piece for connection to the stirring unit, and wherein the stirring unit has a connecting counterpart for connection to the connecting piece of the coupling element, wherein, in a connected state, the connecting piece and the connecting counterpart are configured to transmit a driving force of a first fluid and at least one further fluid.

In particular, the connecting piece may comprise a connection point of the first fluid path, a connection point of the further fluid path, and the second connecting element.

This allows the system to be designed to be more user-friendly and space-saving.

According to embodiments of the invention, the aforementioned task is further solved through the use of a coupling element according to the present disclosure for coupling a drive element to a stirring unit for a fluid container, in particular to a stirring unit for a reactor for chemical reactions and/or for bioprocess engineering, preferably to a stirring unit for a bioreactor.

This way, existing laboratory equipment can be retrofitted. In particular, existing drive systems and their software can continue to be used, while at the same time avoiding costly modifications to the drive control system. Preferably, it can be used in connection with gas fermentation, a fluid contact supply for extraction in a biological or chemical process, or a process in the food industry.

A fluid path can be configured as a channel or cavity, whereby a fluid path can have one or more elements, channels, or cavities connected to each other in terms of fluid flow. Preferably, a fluid path has a first connection point and a second connection point, whereby a fluid flow can be applied between the first connection point and the second connection point.

The middle part of the base body has a first connecting element and a second connecting element, each of which is configured to transmit a driving force. The first connecting element is preferably configured for mechanical connection, in particular for detachable form-fitting connection with a connecting counterpart of the drive element. Examples of a connecting element are a thread, a bayonet lock, a flange coupling, a polygonal bolt, a clamping element, a rough surface for frictional contact, or the like. The first connecting element and the second connecting element may each be arranged at a distal end of the middle part.

The middle part is at least partially arranged in the interior of the outer part, and the interior space is configured such that a free area of the interior space surrounds the middle part. In addition, the free area of the interior space of the outer part and the first cavity of the middle part together form at least part of the first fluid path. For this purpose, the interior space of the outer part preferably has a shape such that, when the middle part is received by the interior space, a free space remains which surrounds the middle part along a possible direction of rotation of the middle part relative to the outer part and which is connected to the first cavity of the middle part in terms of fluid flow. This means that the first fluid path can be flowed through with the first fluid regardless of the rotational position of the middle part relative to the outer part.

The interior space of the outer part can be configured such that a first free area together with the first cavity of the middle part at least partially forms the first fluid path, and such that at least one further free area together with the at least one further cavity of the middle part at least partially forms at least one further fluid path. Thus, both the first fluid path and the further fluid path can be flowed through independently of the rotational position of the middle part relative to the outer part.

A “stirring unit” preferably has a fluid path for conducting a first fluid and is configured to be used together with a fluid container for a further fluid. In particular, the fluid path of the stirring unit is at least partially permeable to the first fluid, so that the stirring unit can be used to bring the first fluid into contact with the further fluid in the fluid container. The fluid path of the stirring unit may comprise a first section, a second section, and a third section, wherein the first section is configured to conduct the first fluid to the second section, wherein the second section comprises a material permeable to the first fluid, for example a permeable membrane, and wherein the third section is configured to conduct a portion of the first fluid that has not been passed through the material of the second section. The membrane may be configured for convective fluid transport or for diffusive fluid transport.

The stirring unit may comprise at least one further fluid path or further fluid path sections which are configured to conduct a mixture of the first fluid with a further fluid and/or further fluids. Thus, several fluids can be brought into contact with the fluid of a fluid container in parallel through the stirring unit. A mixture of an introduced fluid with the fluid of the fluid container can also be discharged from the fluid container. Preferably, the stirring unit has a first connection for connection to the first fluid path of the coupling element and a second connection for connection to a second fluid path of the coupling element.

Preferably, the stirring unit is configured to be movable in a fluid container in order to support mixing or dispersion of the first fluid in the other fluid of the fluid container or to achieve as homogeneous a distribution of the first fluid as possible over the volume of the other fluid. For this purpose, the stirring unit may have a connecting element for receiving a driving force. In particular, the stirring unit may be configured as disclosed in publication WO 2021/152128 A1.

Containers commonly used in chemical and bioprocess engineering can be used as fluid container, whereby these are intended for multiple or single use. A non-exhaustive list of examples includes stirred tank reactors, bubble column reactors, fixed bed reactors, fluidized bed reactors, membrane bioreactors, photobioreactors, reactors for tissue engineering such as roller bottles, flasks, and wave bags. The fluid container may be configured as disclosed in publication WO 2021/152128 A1.

The first fluid that can be conveyed through the first fluid path of the coupling element and/or the further fluid that can be conveyed through at least one further fluid path of the coupling element may contain at least one element, in particular one element from the list: a gas, an oil or extraction agent, n-octane, n-hexane, cyclohexane, ethyl acetate, n-hexadecane, tetradecane, ethyl oleate, 1-decene, 1-dodecene, n-nonane, dioctyl ether, limonene, undecane, cyclooctane, p-cymene, 2-undecanone, 1-octene, n-decane, oleyl alcohol, methyl tert-butyl ether (MTBE), capric acid ethyl ester, a nutrient solution, PBS (phosphate-buffered saline), brilliant green bile lactose broth, casein soy peptone broth (CASO), DMEM (Dulbecco's Modified Eagle's Medium), E-MEM (Eagle's minimal essential medium), meat water, Ham's F12, LB medium, minimal medium 9, RPMI, SOB medium, TY medium, YPD medium, HPL (human platelet lysate).

Various embodiments of the coupling element, the system, and the use are described below, whereby the individual embodiments apply independently of each other to the coupling element, the system, and the use. In addition, the individual embodiments can be combined with each other as desired.

In one embodiment of the coupling element, a first free area of the interior of the outer part and the first cavity of the middle part together form at least part of the first fluid path, and a further free area of the interior of the outer part and the at least one further cavity of the middle part together at least partially form the at least one further fluid path, wherein the first free area of the interior and the further area of the interior are separated from each other in terms of fluid flow by a sealing element.

Thus, the outer part and the middle part form the fluid paths and separate cannulas or tubes are not required.

The coupling element can be configured such that the first area of the interior at least partially surrounds the middle part and such that the further area of the interior at least partially surrounds the middle part. Thus, the fluid path can be flowable with the first fluid independently of the rotational position of the middle part relative to the outer part.

Preferably, the sealing element is a shaft sealing element that ensures a flow-tight connection between the outer part and the middle part while allowing the middle part to rotate relative to the outer part. An example of such a shaft sealing element is a shaft sealing ring. The sealing element can serve as a bearing element between the outer part and the middle part.

The sealing element can contain a plastic, for example Teflon (PTFE), in particular a plastic lip or a plastic coating. Preferably, the sealing element is configured to seal the connection between the outer part and the inner part, wherein the inner part moves at a speed from 0 to 1500 revolutions per minute relative to the outer part, and wherein the sealing element is exposed to a pressure from 0 to 10 bar, in particular a pressure from 0 to 3 bar.

The first fluid path can be separated from the environment of the coupling element by a first sealing element, and the at least one further fluid path can be separated from the environment of the coupling element by a second sealing element. Here, the first sealing element and the second sealing element are shaft sealing elements.

In one embodiment of the coupling element, the first fluid path has a first connection point and a second connection point, the first connection point is located on the outer part, and the second connection point is located on the middle part and, together with the second connecting element for transmitting a drive force, forms a connecting piece for transmitting a drive force and the first fluid.

Thus, the connecting piece can be used to transmit both force and fluid to or from a stirring unit. This allows for a compact and space-saving design of the coupling element. At the same time, user-friendliness is also increased.

In one embodiment of the coupling element, the first cavity and the at least one further cavity are configured essentially as channels and extend essentially parallel to each other in a main direction of the middle part.

This allows the middle part to be formed in one piece and the cavities to be easily created by drilling holes.

In one embodiment of the coupling element, the first cavity of the middle part at least partially surrounds the at least one further cavity of the middle part.

This allows a fluid connection to a stirring unit to be easily and securely ensured independently of rotation of the middle part.

In one embodiment of the coupling element, the middle part has a shaft with a cavity and an inner part with a bore, the inner part is received in the cavity of the shaft in such a way that the cavity is divided into a first cavity region and a second cavity region, wherein the first cavity region corresponds to the first cavity of the middle part for the first fluid path and wherein the second cavity region, together with the bore of the inner part, forms the further cavity of the middle part for the at least one further fluid path.

Concentric holes in one-piece metal components are difficult to produce, especially when the holes have a large length-to-diameter ratio, for example, significantly greater than 30. Designing the middle part as a multi-piece component with parts arranged inside each other offers a simple and cost-effective solution for making the first fluid path and a further fluid path concentric. Arranging the fluid paths concentrically in turn offers the advantage of a space-saving connection with a moving part, such as a stirring unit according to the present disclosure.

The inner part can be rotatably mounted to the shaft. Alternatively, the shaft and the inner part can be connected to each other in a rotationally fixed manner. Preferably, the first cavity region and the second cavity region are separated from each other in terms of fluid flow by a sealing element. If the inner part is rotatably mounted by the shaft, the sealing element is preferably a shaft sealing element.

In one embodiment of the coupling element, the first fluid and/or the further fluid is a gas.

Components with moving parts that are configured to transfer a fluid are generally more difficult to seal when the fluid is a gas than when it is a liquid. This is particularly the case when the component in question is configured for rotational movement. The coupling element thus offers a wider range of possible applications and is particularly suitable for certain biological processes in which a gas is to be transferred.

In one embodiment of the coupling element, the first fluid path and the at least one further fluid path are configured to convey fluids in opposite directions relative to each other.

This allows, for example, a stirring unit to be supplied with a fluid and then excess fluid to be discharged.

Alternatively, the coupling element can be configured so that the first fluid path and the at least one additional fluid path conduct fluids in the same direction. This allows a stirring unit to be supplied with several fluids at the same time or several fluids to be discharged from the stirring unit.

The first fluid path may have a first connection point and a second connection point, and the at least one further fluid path may have a third connection point and a fourth connection point, wherein the first connection point, the second connection point, the third connection point, and the fourth connection point are each configured for introducing and/or discharging a fluid. This allows for the following configurations:
simultaneous introduction of a fluid via the first fluid path and discharge of another fluid via the further fluid path,
simultaneous introduction of a fluid via the first fluid path and introduction of another fluid via the other fluid path,
simultaneous discharge of a fluid via the first fluid path and discharge of another fluid via the other fluid path,
simultaneous discharge of a fluid via the first fluid path and introduction of another fluid via the further fluid path.

The coupling element may have dimensions, for example a length, a width, and/or a diameter, which have been selected depending on the size or volume of a fluid container. The coupling element is compact in design and the base body of the coupling element can have a total length in the range from 0 to 40 cm, in particular in the range from 0 to 35 cm, preferably in the range from 0 to 30 cm, more preferably in the range from 5 to 25 cm, and particularly preferably in the range from 10 to 20 cm. Beside it, the base body may have a width and/or diameter that has been selected depending on the drive force to be transmitted. For example, the base body may have a width in the range from 0 to 10 cm, in particular in the range from 2 to 8 cm, preferably in the range from 3 to 6 cm.

An example of a coupling element has a base body, wherein the outer part of the base body is essentially cylindrical in shape, has a length of approximately 130 mm and a diameter of approximately 42 mm, and wherein the middle part of the base body is essentially cylindrical in shape, has a length of approximately 178 mm and a diameter of approximately 20 mm. Such a coupling element is particularly compact in design and can be used in conjunction with a fluid container having a volume in the range from 0.2 to 10 L and a correspondingly dimensioned stirring unit.

The coupling element may be configured such that the base body has at least one adaptation element, whereby the shape of the adaptation element is adapted to the shape of the outer part and/or the middle part, and whereby the shape of the adaptation element is also adapted to the shape of a fluid container and/or to the shape of a cover element of a fluid container. This allows the coupling element to be used for a wider variety of fluid containers, in particular for fluid containers of different sizes. For example, the shape of the adapter element can be adapted to an opening in a cover element for a fluid container, wherein the volume of the fluid container is equal to or greater than 0.2 L, in particular equal to or greater than 0.25 L, preferably in the range from 0.5 to 50 L, more preferably in the range from 2 to 10 L, possibly equal to or greater than 50 L.

In one embodiment of the system, the stirring unit has a fluid path, and the first fluid path of the coupling element, the fluid path of the stirring unit, and the at least one further fluid path of the coupling element together form a conveying path for a gas or a liquid.

Thus, a first fluid can be conveyed through the coupling element via the first fluid path to the stirring unit, the first fluid can then be transferred through the stirring unit via the fluid path of the stirring unit into a second fluid of a fluid container, and a residual portion of the first fluid, possibly mixed with a portion of the second fluid, can be conveyed out of the stirring unit via the second fluid path of the coupling element.

This means that the system is configured for the simultaneous, separate conveyance of a first fluid and a second fluid, whereby the stirring unit can be set in motion simultaneously by means of power transmission.

In one embodiment of the system, a drive element, a first fluid unit, and at least one further fluid unit are provided, the drive element is connectable to the first connecting element of the coupling element, the first fluid unit is connectable to the first fluid path of the coupling element, and the at least one further fluid unit is connectable to the at least one further fluid path of the coupling element.

This allows a first fluid to be transferred between the first fluid unit and the stirring unit, a second fluid to be transferred between the second fluid unit and the stirring unit, and a drive force to be transferred from the drive element to the stirring unit at the same time.

In a corresponding embodiment of the use, it is provided that a driving force is transmitted from the coupling element to the stirring unit and at least two fluids are transmitted between the coupling element and the stirring unit simultaneously.

In one embodiment of the use, it is provided that the first fluid is conveyed through the first fluid path at a pressure in the range from 0 to 10 bar, in particular in the range from 0 to 3 bar, and/or that the further fluid is conveyed through the at least one further fluid path at a pressure in the range from 0 to 10 bar, in particular in the range from 0 to 3 bar.

Such pressure conditions enable a fluid supply that is desirable for chemical or biochemical reactions. For this purpose, at least one sealing element of the coupling element can be made of a material that can withstand a pressure in the range from 0 to 10 bar, in particular in the range from 0 to 3 bar.

Preferably, the first fluid path and/or the at least one further fluid path of the coupling element is configured to conduct a fluid, in particular a gas, which has a temperature of at least 100 °C, in particular at least 121 °C, preferably at least 150 °C. This allows the coupling element to be disinfected and/or sterilized and also to conduct fluids that are suitable for disinfecting other components of the system. For this purpose, the base body or parts of the base body may be made of stainless steel or PEEK. In addition, at least one of the sealing elements of the coupling element can be made of a temperature-resistant material such as metal or plastic, for example aluminum, stainless steel, Teflon, or others.

In one embodiment of the use, it is provided that the drive element is coupled to the stirring unit by the coupling element, and that the stirring unit rotates at a speed in the range from 0 to 1,500 revolutions per minute, in particular in the range from 0 to 1,500 revolutions per minute, preferably in the range from 0 to 1,000 revolutions per minute, more preferably in the range from 0 to 600 revolutions per minute, in particular preferably in the range from 100 to 600 revolutions per minute, and especially preferably in the range from 0 to 200 revolutions per minute.

The speed or rotational speed at which the stirring unit is driven usually depends on the individual application. Parameters such as the type and composition of the fluid in the fluid container, the gas consumption of biological cells, the dimensions or volume or diameter of the fluid container and the stirring unit itself may be taken into account when selecting the drive speed. For example, in the case of fluid containers with a relatively small diameter, significantly higher rotational speeds can be applied than in the case of fluid containers with a larger diameter in order to achieve a similar circumferential speed in the fluid contained in the respective fluid container.

This means that the coupling element or system can be used at speeds ranging from 0 to 1,500 revolutions per minute for a wide variety of fluid containers. A speed in the range from 0 to 1,000 revolutions per minute is advantageous for cell cultures with relatively high gas consumption. For animal cell cultures, a speed in the range from 0 to 200 revolutions per minute is preferred, while for fermentation processes involving microorganisms, a speed in the range from 100 to 600 revolutions per minute is desirable. A speed in the range from 0 to 600 revolutions per minute can be used for both animal cell cultures and fermentation processes involving microorganisms.

### BRIEF DESCRIPTION OF THE DRAWINGS

Further features and advantages of the coupling element, the system, and its use can be seen from the following description of embodiments, with reference to the accompanying drawing.

In the drawings,

FIG. 1 shows a first embodiment of a coupling element;

FIG. 2a shows a second embodiment of a coupling element in a sectional view;

FIG. 2b the second embodiment of a coupling element in a side view;

FIG. 2c the second embodiment of a coupling element in a perspective view; and

FIG. 3 an embodiment of a system.

### DETAILED DESCRIPTION OF ILLUSTRATIVE EMBODIMENTS

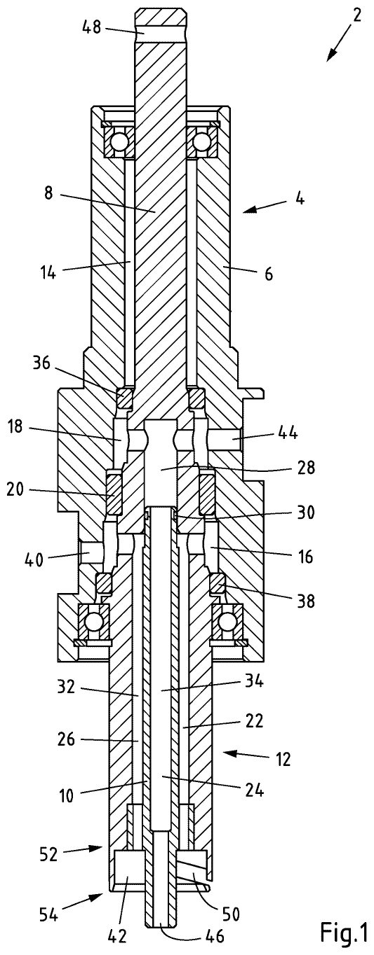

FIG. 1 shows a first embodiment of a coupling element 2 for coupling a drive element to a stirring unit. The coupling element 2 has a base body 4 with an outer part 6, a shaft 8, and an inner part 10, wherein the shaft 8 and the inner part 10 together form a middle part 12.

The outer part 6 is essentially cylindrical in shape and has an interior space 14. The middle part 12 or the shaft 8 and the inner part 10 together are rotatably mounted in the interior space 14 of the outer part 6. The interior space 14 of the outer part 6 and the outer wall of the middle part 12 or the shaft 8 form a first free area 16 and a second free area 18, wherein the first free area 16 and the second free area 18 surround the middle part 12. The first free area 16 of the interior space 14 and the second area 18 of the interior space 14 are separated from each other in terms of flow by a first sealing element 20.

Shaft 8 has a cavity 22 and the inner part 10 has a bore 24. The inner part 10 is received in the cavity 22 of the shaft 8 in such a way that the cavity 22 is divided into a first cavity region 26 and a second cavity region 28. The first cavity region 26 and the second cavity region 28 are separated from each other in terms of fluid flow by a second sealing element 30.

The first cavity region 26 of the shaft 8 and the first free area 16 of the interior space 14 of the outer part 6 together form a first fluid path 32. The bore 24 of the inner part 10, the second cavity region 28 of the shaft 8 and the second free area 18 of the interior space 14 of the outer part 6 together form a second fluid path 34.

A third and a fourth sealing element 36, 38 serve to seal the interior space 14 of the outer part 6.

The fact that the middle part 12 is rotatably mounted in the interior space 14 of the outer part 6, and that the first free area 16 and the second free area 18 of the interior space 14 surround the shaft 8, means that the first fluid path 32 and the second fluid path 34 can be flowed through independently of the rotational position of the middle part 12 relative to the outer part 6.

The first fluid path 32 has a first connection point 40 and a second connection point 42, wherein the first connection point 40 is arranged on the outer part 6, and wherein the second connection point 42 is an opening of the first cavity region 26 of the shaft 8. The second fluid path 34 has a third connection point 44 and a fourth connection point 46, wherein the third connection point 44 is arranged at the outer part 6, and wherein the fourth connection point 46 is an opening of the bore 24 of the inner part 10.

The shaft 8 has a first connecting element 48 for transmitting a driving force and a second connecting element 50 for transmitting a driving force, wherein the second connecting element 50 is arranged at a distal end 52 of the shaft 8 and is configured as a bayonet lock. The second connection point 42 of the first fluid path 32 and the fourth connection point 46 of the second fluid path 34 are arranged at the same distal end 52 of the shaft 8 and the middle part, respectively, and together form a connecting piece 54 for transmitting a driving force, a first fluid, and a second fluid.

The base body 4 of the coupling element 2 has a material composition comprising stainless steel. The sealing elements 20, 30, 36, 38 are configured as shaft sealing rings with Teflon. This makes the coupling element 2 suitable for conveying gases at a pressure of up to 10 bar and a temperature of at least 150 °C, whereby the middle part 12 can undergo a rotational movement relative to the outer part 6 at a speed of up to 1,500 revolutions per minute.

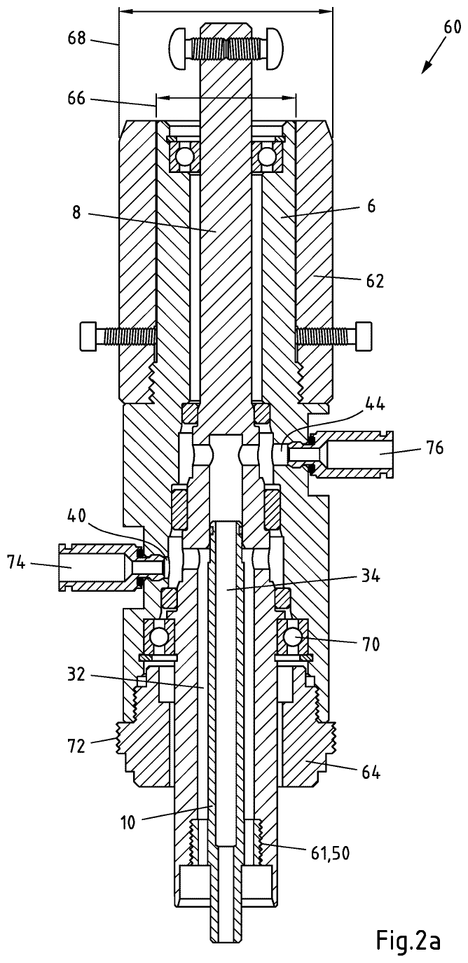

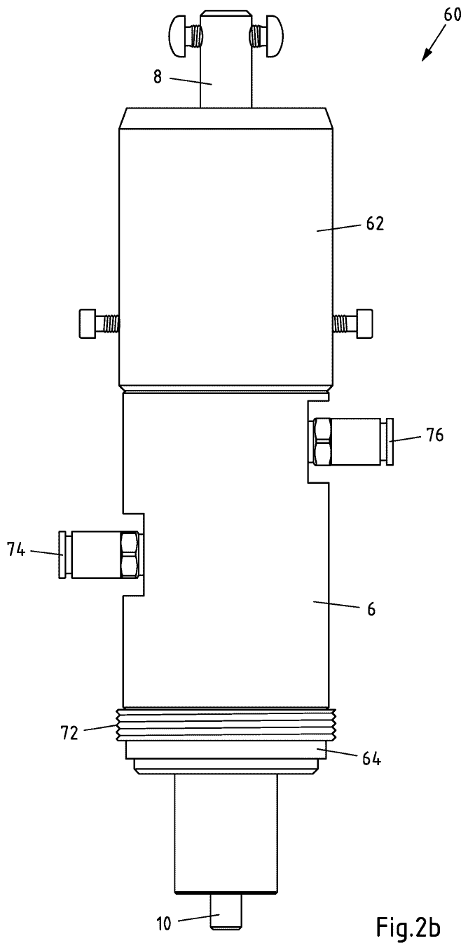

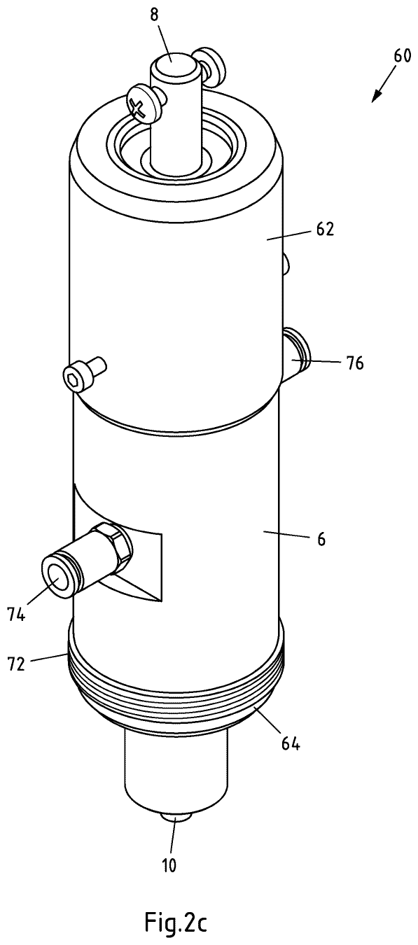

FIGS. 2a, 2b, and 2c show a second embodiment of a coupling element 60 in a sectional view, a side view, and a perspective view, respectively. The coupling element 60 has essentially the same components as the coupling element 2 from FIG. 1, which are marked with the same reference symbols. In contrast to the coupling element 2 from FIG. 1, the second connecting element 50 of the coupling element 60 is configured as a thread 61.

In addition, the coupling element 60 from FIGS. 2a, 2b, and 2c has a first adjustment element 62 and a second adjustment element 64. The first adaptation element 62 is configured as a cylinder with an inner diameter 66 and an outer diameter 68, wherein the inner diameter 66 is adapted to the shape of the outer part 6, and wherein the outer diameter 68 of the first adaptation element 62 is adapted to a housing element of a drive element (not shown) for fastening the coupling element to the housing element.

The second adaptation element 64 is adapted to the shape of the shaft 8 and the outer part 8. In addition, the second adaptation element 64 has a thread 72 that is suitable for connection to a cover element of a fluid container.

The shaft 8 is rotatably mounted to the outer part 6 by a bearing element 70.

The first connection point 40 of the first fluid line 32 and the third connection point 44 of the second fluid line 34 are each connected to a connection element 74, 76 for a gas line.

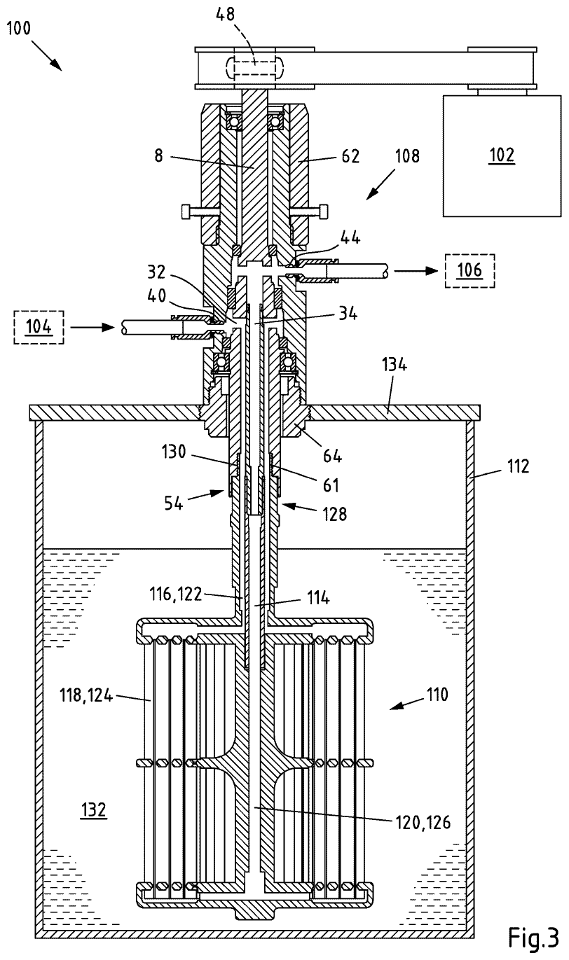

FIG. 3 shows an example of a system 100, wherein the system 100 comprises a drive element 102, a first fluid unit 104, a second fluid unit 106, a coupling element 108, a stirring unit 110, and a fluid container 112. The coupling element 108 is configured as shown in FIGS. 2a, 2b, and 2c.

The drive element 102 is connected to the coupling element 108 via the first connecting element 48 of the shaft 8. The first fluid unit 104 is connected to the first fluid path 32 of the coupling element 108 via the first connection point 40, and the second fluid unit 106 is connected to the second fluid path 34 of the coupling element 108 via the third connection point 44.

The stirring unit 110 has a fluid path 114 with a first section 116, a second section 118, and a third section 120. The first section 116 is configured as a first flow channel 122. The second section 118 has a membrane 124 configured for convective transport of a fluid, and the third section 120 is configured as a second flow channel 126. The stirring unit 110 has a connecting counterpart 128 into which the first channel 122 and the second channel 126 open, with the second channel 126 surrounding the first channel 122. The connecting counterpart 128 also has a thread 130 as a counterpart to the thread 61 of the coupling element 108.

The stirring unit 110 and the coupling element 108 are connected fluidically and mechanically via a connection of the connecting piece 54 to the connecting counterpart 128.

The stirring unit 110 is arranged in the receiving volume of the fluid container 112 and immersed in a liquid 132. The coupling element 108 is connected to a cover element 134 of the fluid container 112 via the second adapter element 64.

1.-15. (canceled)

16. A system comprising:
a coupling element configured for coupling a drive element to a stirring unit,
wherein the coupling element comprises a base body,
wherein the base body has a first fluid path configured for conveying a first fluid and at least one second fluid path configured for conveying a second fluid, wherein the first fluid path and the at least one second fluid path are separated from each other in terms of fluid flow,
wherein the base body comprises an outer part with an interior space and a middle part with a first cavity and with at least one second cavity, wherein the middle part has a first connecting element configured for transmitting a driving force from the drive element and a second connecting element configured for transmitting the driving force to the stirring unit,
wherein the middle part is rotatably mounted to the outer part,
wherein the middle part is arranged at least partially in the interior space of the outer part, and the interior space is configured such that a free area of the interior space surrounds the middle part, wherein the free area of the interior space of the outer part and the first cavity of the middle part together at least partially form the first fluid path,
wherein the at least one second cavity of the middle part at least partially forms the at least one second fluid path, wherein the coupling element comprises a connecting piece configured for connecting to the stirring unit,
wherein the stirring unit has a connecting counterpart configured for connecting to the connecting piece, and
wherein, in a connected state, the connecting piece and the connecting counterpart are configured to transmit the driving force, the first fluid, and the at least one second fluid.

17. The system according to claim 16,
wherein a first free area of the interior space and the first cavity together form, at least partially, the first fluid path, wherein a second free area of the interior space of the outer part and the at least one second cavity together at least partially form the at least one further fluid path, and
wherein the first free area and the second free area are separated from each other in terms of flow by a sealing element.

18. The system according to claim 16,
wherein the first fluid path comprises a first connection point and a second connection point, wherein the first connection point is arranged at the outer part, and
wherein the second connection point is arranged at the middle part and, together with the second connecting element, forms the connecting piece configured for transmitting the driving force and the first fluid.

19. The system according to claim 16, wherein the first cavity and the at least one second cavity are configured as channels and extend parallel to each other in a main direction of extension of the middle part.

20. The system according to claim 16, wherein the first cavity at least partially surrounds the at least one second cavity.

21. The system according to claim 16,
wherein the middle part comprises a shaft with a cavity and an inner part with a bore,
wherein the inner part is received in the cavity of the shaft such that the cavity is divided into a first cavity region and a second cavity region,
wherein the first cavity region corresponds to the first cavity for the first fluid path, and
wherein the second cavity region together with the bore forms the second cavity.

22. The system according to claim 16, wherein the first fluid and/or the second fluid is a gas.

23. The system according to claim 16, wherein the first fluid path and the at least one second fluid path are configured to convey fluids in opposite directions relative to each other.

24. The system according to claim 16,
wherein the stirring unit comprises a fluid path, and
wherein the first fluid path of the coupling element, the fluid path of the stirring unit and the at least one second fluid path of the coupling element together form a conveying path for a gas or a liquid.

25. The system according to claim 16, further comprising:
the drive element;
a first fluid unit; and
at least one second fluid unit,
wherein the drive element is connectable to the first connecting element of the coupling element,
wherein the first fluid unit is connectable to the first fluid path of the coupling element, and
wherein the at least one second fluid unit is connectable to the at least one second fluid path of the coupling element.

26. The system according to claim 16, further comprising:
a cover element of a fluid container,
wherein the coupling element comprises an adaptation element,
wherein a shape of the adaptation element is adapted to a shape of the outer part and/or the middle part of the coupling element,
wherein the shape of the adaptation element is also adapted to a shape of the cover element, and
wherein the coupling element is connected to the cover element via the adaptation element.

27. A method for operating a coupling element, wherein the coupling element comprises a base body, wherein the base body comprises a first fluid path for conveying a first fluid and at least one second fluid path for conveying a second fluid, wherein the first fluid path and the at least one second fluid path are separated from each other in terms of fluid flow, wherein the base body has an outer part with an interior space and a middle part with a first cavity and with at least one second cavity, wherein the middle part has a first connecting element for transmitting a driving force from a drive element and a second connecting element for transmitting the driving force to a stirring unit, wherein the middle part is rotatably mounted to the outer part, wherein the middle part is arranged at least partially in the interior space of the outer part, and the interior space is configured such that a free area of the interior space surrounds the middle part, wherein the free area of the interior space and the first cavity together at least partially form the first fluid path, and wherein the at least one second cavity of the middle part at least partially forms the at least one second fluid path, the method comprising:
transmitting the driving force from the coupling element to the stirring unit; and
simultaneously, transmitting at least two fluids between the coupling element and the stirring unit.

28. The method according to claim 27, wherein the coupling element couples the drive element to the stirring unit for a reactor for chemical reactions and/or for bioprocess engineering.

29. The method according to claim 27,
wherein the first fluid is conveyed through the first fluid path at a pressure in a range from 0 to 10 bar, and/or
wherein the at least one second fluid is conveyed through the at least one second fluid path at a pressure in a range from 0 to 10 bar.

30. The method according to claim 27,
wherein the drive element is coupled to the stirring unit via the coupling element, and
wherein the stirring unit is driven at a speed in a range from 0 to 1,500 revolutions per minute.

31. The method according to claim 30, wherein the stirring unit is driven at the speed in a range from 0 to 200 revolutions per minute.
